(19) United States
(12) Plant Patent Application Publication
(10) Pub. No.: US 2003/0046740 P1
(43) Pub. Date: Mar. 6, 2003

(54) Chrysanthemum plant named 'MARVINE'

(75) Inventor: Robert Noodelijk, Woubrugge (NL)

Correspondence Address: PARKHURST & WENDEL, L.L.P., 1421 PRINCE STREET, SUITE 210, ALEXANDRIA, VA, 22314-2805, US

(73) Assignee: Chrysanthemum Breeders Association N.V.

(21) Appl. No.: 10/175,820

(22) Filed: Jun. 21, 2002

(30) Foreign Application Priority Data
Jun. 28, 2001 (NL) CHR3275

(51) Int. Cl. A01H 5/00

(52) U.S. Cl. PLT/286

(57) ABSTRACT

A Chrysanthemum plant named ‘MARVINE’ characterized by its large sized blooms with purple and white ray-florets and yellow-green disc florets.

### BACKGROUND OF THE INVENTION

‘MARVINE’ is a product of a breeding-program which had the objective of creating new chrysanthemum cultivars with a daisy type flower, a 7 week response and a medium plant height. The new plant of the present invention comprises a new and distinct cultivar of Chrysanthemum plant. ‘MARVINE’ is a seedling from a cross in a breeding program maintained under the control of inventor. The female parent is # 94.1301, the male parent is # 94.1106, both unpatented, unamed seedlings. The parents are not available to inventor for description. The new and distinct cultivar was discovered and selected as a flowering plant within the progeny of the stated cross by Rob Noodelijk in a controlled environment (greenhouse) in Rijsenhout Holland in April 1997. The first act of asexual reproduction of ‘MARVINE’ was accomplished when vegetative cuttings were taken from the initial selection in June 1997 in a controlled environment in Rijsenhout Holland.

### SUMMARY OF THE INVENTION

The present invention is a new and distinct variety of chrysanthemum bearing large sized blooms with purple and white rayflorets and yellow-green disc florets.

### BRIEF DESCRIPTION OF THE DRAWINGS

The present invention of a new and distinct variety of chrysanthemum is shown in the accompanying drawings, the color being as nearly true as possible with color photographs of this type.

FIG. 1 shows a plant of the cultivar in fall bloom.

FIG. 2 shows the various stages of bloom of the new cultivar.

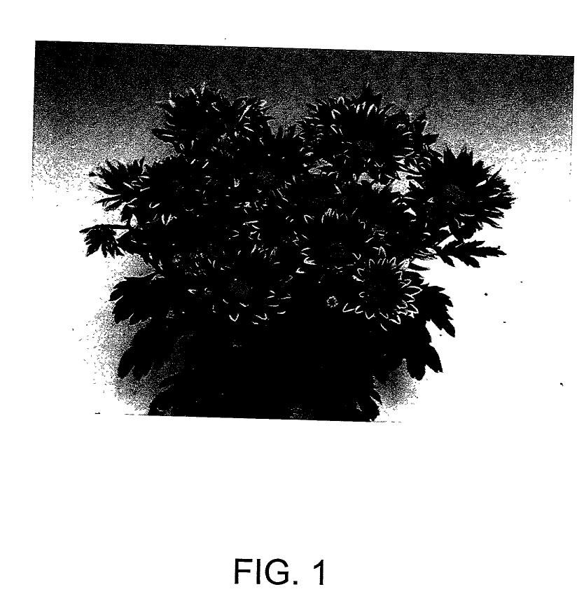

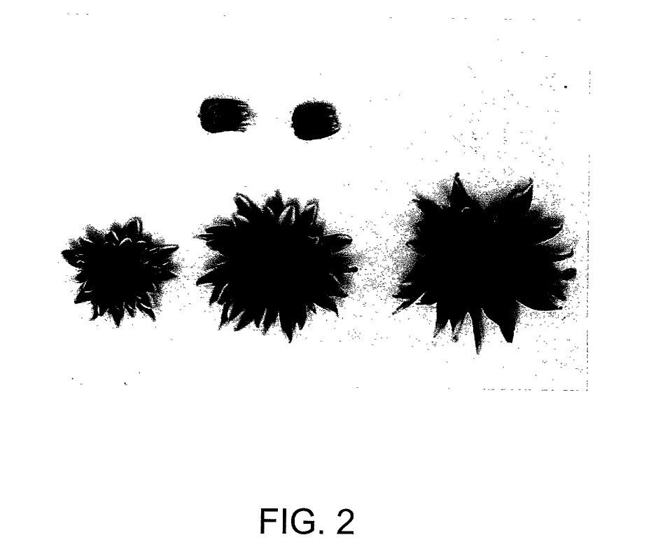

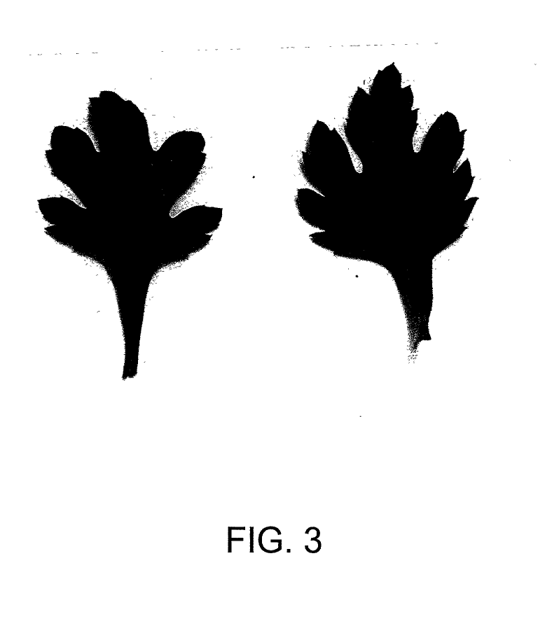

FIG .3 shows the foliage of the new cultivar

### DESCRIPTION OF THE INVENTION

This new variety of chrysanthemum is of the botanical classification chrysanthemum morifolium. The observations and measurements were gathered from plants grown in a greenhouse in Rijsenhout Holland in a photo-periodic controlled crop under conditions generally used in commercial practice. The greenhouse temperatures during this crop were at day-time between 18° C. and 25° C. and at night 20° C. The photo-periodic response time in this crop was 48 days after an average of eight long days. After this long day period to flowering growth retardants were applied 6 times in an average dose of 1.5 gram/liter water. The plants were observed (directly) during the flowering of this crop. No tests were done on disease or insect resistance or susceptibility. No tests were done on cold or drought tolerance. This new variety produces large sized blooms with purple and white ray-florets and yellow-green disc-florets blooming on the plant for 5 weeks. This new variety of chrysanthemum has been found to retain its distinctive characteristics throughout successive propagations however the phenotype may vary significantly with variations in environment such as light intensity and temperature. To show the phenotype as described ‘MARVINE’ can be planted without assimilation lightning (high pressure sodium lamps) between week 50 and week 40 of the next year under greenhouse conditions in Holland. With assimilation lightning (minimum level 2500 lux) it can be planted year round under greenhouse conditions in Holland.

From the cultivars known to inventor the most similar existing cultivar in comparison to ‘MARVINE’ is ‘Tattoo time’. When ‘Tattoo time’ and ‘MARVINE’ are being compared the following differences are noticed: The difference of ‘Tattoo time’ and ‘MARVINE’ are (1) flower size and (2) flower color.

The following is a description of the plant and characteristics that distinguish ‘MARVINE’ as a new and distinct variety. The color designations are taken from the plant itself. Accordingly, any discrepancies between the color designations and the colors depicted in the photographs are due to photographic tolerances. The color chart used in this description is: The Royal Horticultural Society color chart, edition 1995.

Botanical Description of CULTIVAR ‘MARVINE’

BUD Size Small;cross-section 1.2 cm, height 0.8 cm Outside Color.—Involucral bracts.—2 rows, length 7 mm, width 3 mm Involucral bracts among disc-florets Not present Involucral bracts color Yellow-green 144 B

BLOOM.—Type Daisy Height Medium high Size Large Fully Expanded 7.0 cm Number of blooms per branch Approx. 2-3 blooms per branch Performance on the plant 5 weeks Seeds Produced in small quantities, oval shaped, greybrown 199A, 2 mm in length. Fragrance Typical chrysanthemum

COLOR Center of the flower (disc-florets) Immature yellow-green 144B Mature yellow-green 144C Color of upper surface of the ray-florets Red-purple 64A, white 155D at the tip Color of the lower surface of the ray-florets Red-purple 57C Tonality from Distance A pot mum with purple and white flowers and a yellow-green disc Color of the surface of the ray-florets after aging of the plant

Ray florets.—Texture Upper and under side smooth Number 24-26Cross-section Convex Longitudinal axis of majority Reflexing; twisted when mature Length of corolla tube Very short Ray-floret length 3.0-3.5cm Ray-floret width 0.9-1.2cm Ratio length/width Low Shape of tip Pointed

Disc florets.—Disc diameter 1.5 cm Distribution of disc florets Numerous, clearly visible at all stages of flowering Shape Tubular Color Yellow-green 144C Receptacle shape Conical raised

Reproductive Organs.—Stamen (present in disc florets only) Thick, 3 mm in length Number of stamen 4 Stamen color Yellow-green Pollen Appears at a very late stage Pollen color Yellow 12A Styles (present in both ray and disc Thin florets) Style color Yellow-green Style Length 5 mm Stigmas Yellow-green Stigma Width 2 mm Ovaries Enclosed in calyx

PLANT Form A pot mum meant for indoor use Growth habit Spreading Growth rate Rapid Height 28.0-30.0cm Width 26.0 cm Stem Color Yellow-green 144 B Stem Strength Weak Stem Brittleness Absent Stem Anthocyanin Coloration Absent Length of lateral branch From top to bottom 12.0-13.0 cm Lateral branch color Yellow-green 144B Lateral branch, attachment Brittle Branching (average number of Good with 4-5 breaks after pinching lateral branches) Peduncle length 3.0-4.0 cm Peduncle color Yellow-green 144 B Flowering Response(photo-periodic 48 Days controlled crop, not natural season

FOLIAGE Color mature Upper side green 137A Under side green 138B Color immature Upper side green 137A Under side green 138 B Size Medium; length 7.0 cm, width 5.0 cm Quantity (number per lateral 5-6 branch) Shape Oval Texture upper side Glabrous Texture under side Pubescent Venation arrangement Palmate Shape of the margin Serrated Shape of Base of Sinus Between Lateral Lobes Round Margin of Sinus Between Lateral Lobes Diverging Shape of Base Acuse Apex Mucronate

Differences with the comparison varieties
When grown under the same conditions

| | ‘Marvine’ | ‘Tattoo time’ |
|---|---|---|
| Flower size | 7.0 cm | 5.5-6.0 cm |
| Flower color | Red-purple 64A and clearly | Red-purple 59A and a little |
| | white 155D at the tips of the | white at the tips of the ray- |
| | ray-florets. | florets. |

I claim:

1. A new and distinct variety of chrysanthemum plant as described and illustrated.
